(19) United States
(12) Patent Application Publication
(10) Pub. No.: US 2022/0023031 A1
(43) Pub. Date: Jan. 27, 2022

(54) SEPTAL-PERICARDIAL HEART VALVE IMPLANT ANCHOR

(71) Applicant: Cardiosolutions, Inc., West Bridgewater, MA (US)

(72) Inventors: Thomas C. Piemonte, Dover, MA (US); Jonathan E. Wilson, Mattapoisett, MA (US); Richard Rodgers, Hudson, MA (US)

(73) Assignee: Cardiosolutions, Inc., West Bridgewater, MA (US)

(21) Appl. No.: 17/381,663

(22) Filed: Jul. 21, 2021

Related U.S. Application Data
(60) Provisional application No. 63/054,503, filed on Jul. 21, 2020.

(51) Int. Cl. A61F 2/24

(52) U.S. Cl. CPC A61F 2/24; A61F 2220/0016; A61F 2210/0004

(57) ABSTRACT

Systems and methods for reversing remodeling of the heart caused by mitral regurgitation are disclosed. An exemplary method may comprise installing a valve implant to at least partially restrict mitral regurgitation, and anchoring the valve implant using one or more anchors that are each attached to the wall of the heart. The anchors may include a pre-tensioned elastic material coated with a slow-absorbable or dissolvable coating configured to release the tension of the pre-tensioned elastic material at an appropriate time to promote reshaping of the heart.

### CROSS-REFERENCE TO RELATED APPLICATIONS

This application claims priority to U.S. Provisional Patent Application No. 63/054,503, filed Jul. 21, 2020 and entitled, “SEPTAL-PERICARDIAL HEART VALVE IMPLANT ANCHOR”, which is incorporated herein by reference in its entirety.

### TECHNICAL FIELD

The present disclosure relates to the repair and/or correction of dysfunctional heart valves, and more particularly pertains to heart valve implants and systems and methods for delivery and implementation of the same.

### BACKGROUND

Heart valve implants and spacers require anchoring to maintain position. Position-maintaining anchoring methods are described in U.S. Patent Application Publication No. 2015/0073547 A1 entitled “Balloon Mitral Spacer,” which is incorporated herein by reference in its entirety. Current anchoring methods attach the spacer to the arterial wall, typically near the apex, or to the interventricular septum. The anchors are used to hold the implant or spacer in position after implantation.

Damaged heart valves that are unable to fully close can result in leakage. Such leakage can result in reduced blood flow. Mitral regurgitation is one common example of this type of failure. When mitral regurgitation occurs, the structure of the heart remodels (i.e., expands in size) as a result of persistent strain on the heart. Remodeling occurs as a result of the body's attempt to compensate for the reduced blood flow resulting from the leakage. Remodeling can result in a comparable expansion in the width of the mitral valve resulting in further leakage. As such, initial remodeling can lead to further remodeling of the heart. Eventually, the mitral valve is so enlarged and so damaged that valve replacement is not viable.

Thus, a heart valve implant is needed that prevents and/or reverses heart remodeling.

### SUMMARY

This summary is provided to comply with 37 C.F.R. § 1.73, requiring a summary of the invention briefly indicating the nature and substance of the invention. It is submitted with the understanding that it will not be used to interpret or limit the scope or meaning of the present disclosure.

A mitral valve implant is provided. The mitral valve implant includes a valve body configured to be disposed within the mitral valve and at least partially restrict mitral regurgitation, and one or more anchors, each with a first and second end, wherein the first end is attached to the valve body and the second end is attached to the wall of the heart, and wherein the one or more anchors are configured to reverse heart remodeling associated with mitral regurgitation.

According to certain embodiments, the second end of at least one of the one or more anchors is attached to the apex of the heart.

According to certain embodiments, the second end of at least one of the one or more anchors is attached to the septum of the heart.

According to certain embodiments, the second end of at least one of the one or more anchors is attached to the pericardial wall of the heart.

According to certain embodiments, at least one of the one or more anchors comprises an elastic member.

According to certain embodiments, the elastic member is pre-tensioned to a first length accommodating a first shape of the heart.

According to certain embodiments, the elastic member comprises a coating in a material configured to be at least one of slow-absorbable or dissolvable in the human body, and absorbing or dissolving the coating releases tension in the elastic member.

According to certain embodiments, the coating is configured to be absorbed or dissolved in a predetermined time.

According to certain embodiments, releasing the tension in the elastic member comprises changing the length of the elastic member from the first length to a second length to accommodate a second shape of the heart.

A method of reversing remodeling of the heart in provided. The method includes inserting, within the mitral valve, a valve body configured to least partially restrict mitral regurgitation, and attaching one or more anchors, each with a first and second end, wherein the first end is attached to the valve body and the second end is attached to the wall of the heart, and wherein the one or more anchors are configured to reverse heart remodeling associated with mitral regurgitation.

According to certain embodiments, attaching one or more anchors comprises attaching a second end of at least one of the one or more anchors to an apex of the heart.

According to certain embodiments, attaching one or more anchors comprises attaching a second end of at least one of the one or more anchors to a septum of the heart.

According to certain embodiments, attaching one or more anchors comprises attaching a second end of at least one of the one or more anchors to a pericardial wall of the heart.

According to certain embodiments, at least one of the one or more anchors comprises an elastic member.

According to certain embodiments, pre-tensioning the elastic member to a first length accommodating a first shape of the heart.

According to certain embodiments, the elastic member comprises a coating in a material configured to be at least one of slow-absorbable or dissolvable in the human body, and wherein absorbing or dissolving the coating releases tension in the elastic member.

According to certain embodiments, the coating is configured to be absorbed or dissolved in a predetermined time.

According to certain embodiments, releasing the tension in the elastic member comprises changing the length of the elastic member from the first length to a second length to accommodate a second shape of the heart.

### BRIEF DESCRIPTION OF THE DRAWINGS

Features and advantages of the claimed subject matter will be apparent from the following description of embodiments consistent therewith, which description should be considered in conjunction with the accompanying drawings, wherein:

FIG. 1A depicts an illustrative cross-sectional view of a normal heart.

FIG. 1B depicts an illustrative cross-sectional view of a remodeled heart with chronic compensation from mitral regurgitation.

FIG. 1C depicts an illustrative cross-sectional view of a remodeled heart with chronic decompensation from mitral regurgitation.

FIG. 2A depicts a cross-sectional view of a heart having an implanted spacer with an anchor located at the apex of the heart in accordance with an embodiment.

FIG. 2B depicts a cross-sectional view of a heart including forces resulting from contraction of the heart in which the spacer of FIG. 2A has been implanted in accordance with an embodiment.

FIG. 3 depicts a cross-sectional view of a heart having an implanted spacer with a cross septal-pericardial anchor in accordance with an embodiment.

FIG. 4 depicts a cross-sectional view of a heart having an implanted spacer with a cross septal-pericardial anchor and an anchor at the apex in accordance with an embodiment.

FIG. 5 depicts a cross-sectional view of a heart including forces resulting from contraction of the heart in which the spacer of FIG. 3 has been implanted in accordance with an embodiment.

FIG. 6A depicts a cross-sectional view of a heart including illustrative anchor connections for the cross septal-pericardial anchor in accordance with various embodiments.

FIG. 6B depicts the expansion of an anchor in accordance an embodiment.

### DETAILED DESCRIPTION

The present description and claims may make use of the terms “a,” “at least one of,” and “one or more of,” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that more than one of such features/elements be present. To the contrary, these terms/phrases only require at least a single feature/element with the possibility of multiple of such features/elements being within the scope of the description and claims.

In addition, it should be appreciated that the following description uses multiple examples for various elements of the illustrative embodiments to further illustrate example implementations of the illustrative embodiments and to aid in the understanding of the mechanisms of the illustrative embodiments. These examples are intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the example provided herein without departing from the spirit and scope of the present invention.

Generally, a heart valve implant consistent with the present disclosure may interact with at least a portion of an existing heart valve to prevent and/or reduce mitral regurgitation and/or heart remodeling. For example, at least a portion of one or more cusps of the heart valve may interact with, engage, and/or seal against at least a portion of the heart valve implant when the heart valve is in a closed condition. The interaction, engagement, and/or sealing between at least a portion of at least one cusp and at least a portion of the heart valve implant may reduce and/or eliminate mitral regurgitation in a heart valve resulting from, for example, providing insufficient sealing, including only a single cusp, e.g., following removal of a diseased and/or damaged cusp, and/or having a ruptured cordae. A heart valve implant, or spacer, consistent with the present disclosure may be used in connection with various additional and/or alternative defects and/or deficiencies. The placement of a heart valve implant is made possible through the use of an anchor. The anchor may be placed in or through the pericardial tissue or in the septum of the heart.

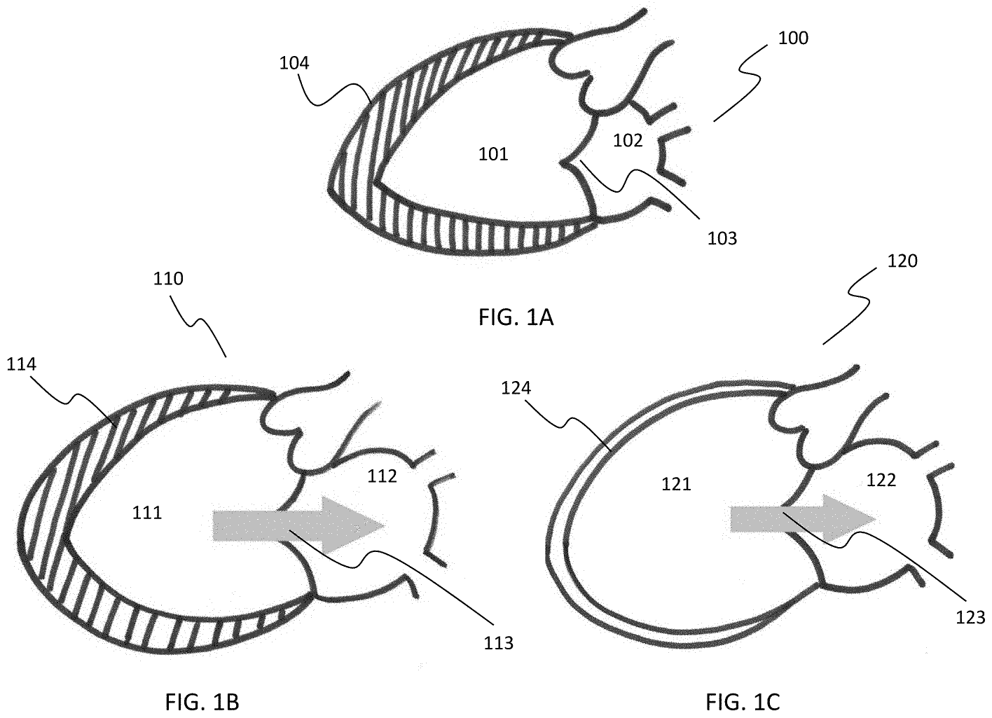

Referring to FIG. 1A, a heart 100 with a normally functioning mitral valve 103 is depicted. As shown in FIG. 1A, the heart 100 has a normally sized left ventricle 101, and the volume of the left atrium 102 and the thickness of the heart wall 104 are also normal. As a result, the heart 100 has normal contractility, wall stress, total stroke volume and forward stroke volume.

FIG. 1B depicts a heart 110 that has remodeled due to mitral regurgitation 113. Specifically, the heart 110 has undergone eccentric hypertrophy leading to an increase in the volume of the left ventricle 111 and the left atrium 112 and an increased wall thickness 114. As a result, the heart 110 has increased preload, contractility, and increased total stroke volume. Hypertrophy of the heart may lead to reduced blood supply to the heart, heart failure, arrhythmia, stroke, ischemic heart disease, and/or cardiac arrest.

FIG. 1C depicts a heart 120 which has alternatively remodeled due to mitral regurgitation 123. Specifically, the heart 120 has decompensated for the mitral regurgitation, leading to a greatly increased left ventricle volume 121 and increased left atrium 122 volume, but a decreased wall thickness 124. As a result, the heart 120 has increased preload, decreased contractility, greatly increased wall stress, and lower total and forward stroke volume. Hypertension of the myocardial wall may lead to similar issues as disclosed above in regards to hypertrophy.

The heart implants disclosed herein not only treat the underlying mitral regurgitation, but also prevent and/or reverse the remodeling of the heart caused by the mitral regurgitation.

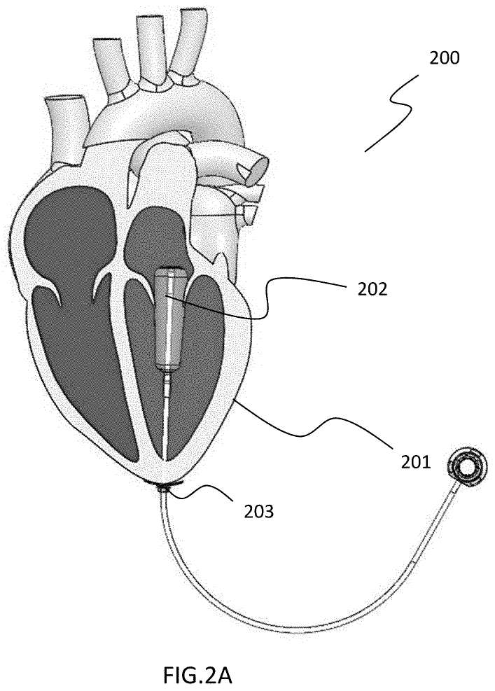

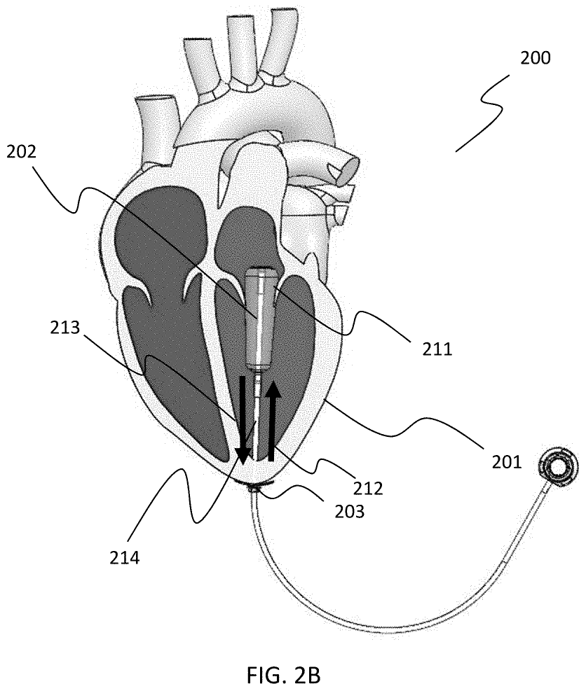

FIG. 2A depicts the application 200 of a mitral spacer 202 in accordance with an embodiment. In some embodiments, the mitral spacer 202 may be anchored near the apex 203 of the heart 201. Anchoring to the apex 203 of the heart 201 may keep the spacer 202 properly positioned during extended use. Furthermore, anchoring to the apex 203 of the heart 201 may produce an observable and significant reverse remodeling of the heart 201. In some cases, reverse remodeling may enable patients to become eligible for heart valve replacement. Reverse remodeling may occur as a result of preventing valve leakage. The prevention of valve leakage may, in turn, remove strain on the heart 201 that occurs when the heart compensates for lost blood flow, thereby providing the heart 201 with an opportunity to recover.

The impact of the use of spacers, as disclosed herein, may be comparable to the use of cardiac resynchronization therapy (CRT). CRT re-coordinates contraction by applying appropriately timed stimuli to the ventricles, through the use of a pacemaker. In either case, these treatments allow the ventricles to close properly and fully at the correct time. Previous attempts at pharmacologic treatment reverse remodeling have had limited success.

Referring to FIG. 2B, the embodiment 200 of FIG. 2A is depicted, along with the forces created by the anchor 214, in accordance with an embodiment. In some embodiments, the pumping action of the heart 201 combined with the action of the mitral valve 211 with respect to the implanted spacer 202, during refilling of the left ventricle, provides a force 212 that pulls the anchor point (i.e., the apex 203 of the heart 201), which prevents expansion 213 of the heart.

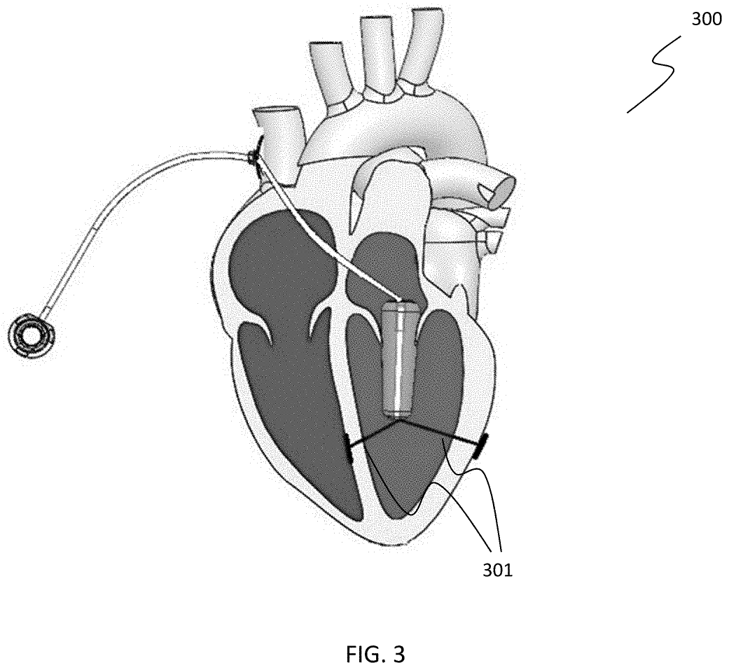

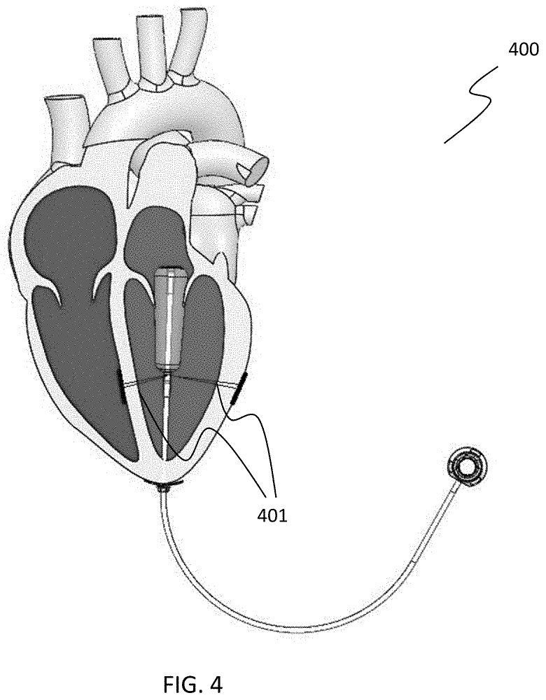

In some embodiments, the location and design of the anchor for the spacer may be chosen to enhance reverse remodeling. In some embodiments, such as depicted in FIG. 3, one or more anchors that provide cross septal—pericardial forces 301 on the heart 300 may aid in reverse remodeling. Such forces would not only aid in supporting the septum and pericardium against the pressure created during pumping, but also aid in their movement inwards, such as during healthy beating. Therefore, the opposite ventricle would also benefit from the more natural movement of the heart.

In some embodiments, the anchor design may include connections to the septum and pericardial wall, as depicted in FIG. 3. Referring to FIG. 4, in some embodiments, the spacer may include anchor points 401 on the septum, the pericardial wall, and the apex of the heart 400.

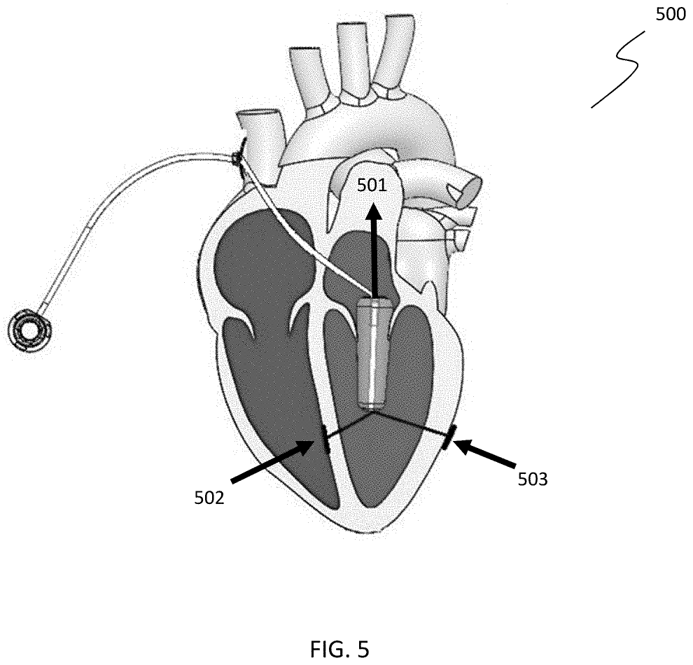

FIG. 5 depicts the forces acting on the heart as a result of anchoring the spacer to the septum and pericardial walls 500, in accordance with an embodiment. The contraction of the heart results in an upward force 501 on the spacer, which in turn pulls in the septal and pericardial anchors 502, 503. These forces 501/502 may counter expansion of the left ventricle as depicted in FIGS. 1B and 1C. In some embodiments, the forces 501/502 may additionally increase contractility by working in tandem with the natural contractions of the heart 500.

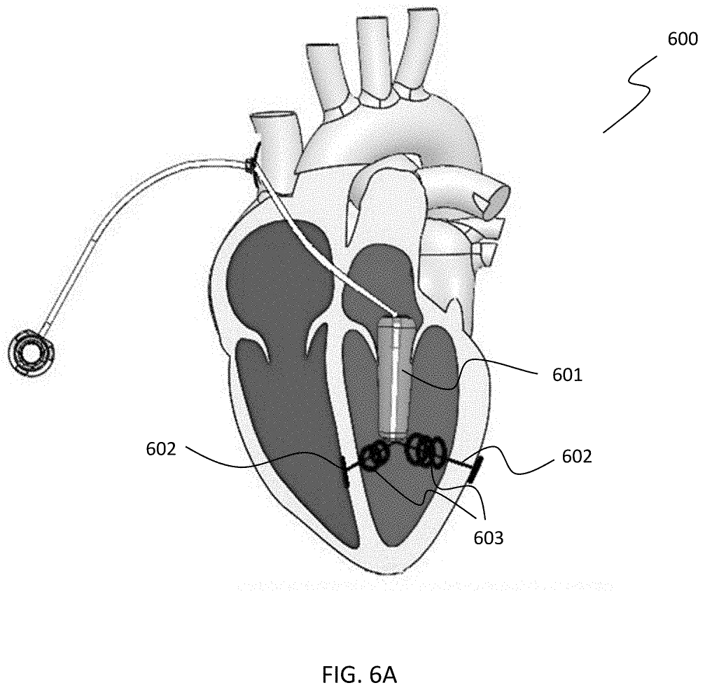

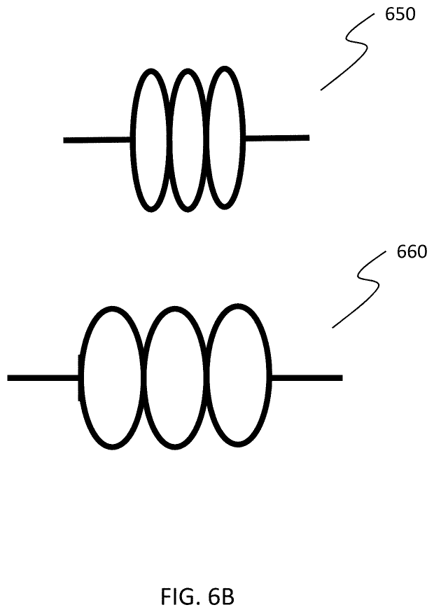

In some embodiments, the anchor connections to the spacer may include elastic members (e.g., springs or expandable materials) to maintain tension as reverse remodeling occurs. FIG. 6A depicts a spacer 601 featuring anchors 602, each anchor comprising a coil 603, in accordance with an embodiment 600. In some embodiments, the elastic member may be pre-tensioned. In some embodiments, the pre-tensioned elastic member may be coated in a slow-absorbable or dissolvable material (e.g. polyglycolic acid, polylactic acid, polydioxanone, caprolactone, and/or catgut). Briefly referring to FIG. 6B, in some embodiments, the coils may be released and allowed to transform from a compressed state 650 to an expanded state 660. Alternatively, in some embodiments, the coils may be released and allowed to transform from an expanded state 660 to a compressed state 650. As remodeling is reversed, the heart may reshape, requiring anchors having different lengths. As such, in such embodiments, the timing of the change in tension may be adjusted to the expected reverse remodeling rate and/or clinical requirements by modifying the composition or characteristics of the coating.

In some embodiments, the anchors may be adjustable surgically by resetting the length of the elastic members through tension. In other embodiments, the anchors may be designed to provide a sliding action (e.g., a piston or comparable assembly). In such embodiments, the anchors may be connected to, for example, an implanted port (not shown) through which injection or removal of saline or other fluids would result in a length adjustment. In such embodiments, adjustments may be performed noninvasively.

In some embodiments, the anchors may retain some elasticity to mimic the functionality of chordae tendineae within the heart. This functionality may further aid in reversing remodeling because the chordae tendineae may be stretched or ruptured as a result of the previous remodeling of the heart.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A mitral valve implant comprising:
a valve body configured to be disposed within a mitral valve of a heart and at least partially restrict mitral regurgitation; and
one or more anchors, each anchor having a first and second end, wherein the first end is attached to the valve body and the second end is configured to be attached to a wall of the heart, wherein the one or more anchors are configured to reverse heart remodeling associated with mitral regurgitation.

2. The implant of claim 1, wherein the second end of at least one of the one or more anchors is configured to be attached to an apex of the heart.

3. The implant of claim 1, wherein the second end of at least one of the one or more anchors is configured to be attached to a septum of the heart.

4. The implant of claim 1, wherein the second end of at least one of the one or more anchors is configured to be attached to a pericardial wall of the heart.

5. The implant of claim 1, wherein at least one of the one or more anchors comprises an elastic member.

6. The implant of claim 5, wherein the elastic member is pre-tensioned to a first length accommodating a first shape of the heart.

7. The implant of claim 6, wherein the elastic member comprises a coating in a material configured to be at least one of slow-absorbable or dissolvable in the human body, and wherein absorbing or dissolving the coating releases tension in the elastic member.

8. The implant of claim 7, wherein the coating is configured to be absorbed or dissolved in a predetermined time.

9. The implant of claim 7, wherein releasing the tension in the elastic member comprises changing a length of the elastic member from the first length to a second length to accommodate a second shape of the heart.

10. A method of reversing remodeling of a heart comprising:
inserting, within the mitral valve of the heart, a valve body configured to at least partially restrict mitral regurgitation; and
attaching one or more anchors, each anchor having a first and second end, wherein the first end is attached to the valve body and the second end is configured to be attached to a wall of the heart, and wherein the one or more anchors are configured to reverse heart remodeling associated with mitral regurgitation.

11. The method of claim 10, wherein attaching one or more anchors comprises attaching a second end of at least one of the one or more anchors to an apex of the heart.

12. The method of claim 10, wherein attaching one or more anchors comprises attaching a second end of at least one of the one or more anchors to a septum of the heart.

13. The method of claim 10, wherein attaching one or more anchors comprises attaching a second end of at least one of the one or more anchors to a pericardial wall of the heart.

14. The method of claim 10, wherein at least one of the one or more anchors comprises an elastic member.

15. The method of claim 14, further comprising pre-tensioning the elastic member to a first length accommodating a first shape of the heart.

16. The method of claim 15, wherein the elastic member comprises a coating in a material configured to be at least one of slow-absorbable or dissolvable in the human body, and wherein absorbing or dissolving the coating releases tension in the elastic member.

17. The method of claim 16, wherein the coating is configured to be absorbed or dissolved in a predetermined time.

18. The method of claim 16, wherein releasing the tension in the elastic member comprises changing a length of the elastic member from the first length to a second length to accommodate a second shape of the heart.
